(19) United States
(12) Patent Application Publication
(10) Pub. No.: US 2021/0227845 A1
(43) Pub. Date: Jul. 29, 2021

(54) PEDIATRIC NUTRITIONAL COMPOSITIONS AND METHODS FOR INFANTS DELIVERED BY C-SECTION

(71) Applicant: MEAD JOHNSON NUTRITION COMPANY, Evansville, IN (US)

(72) Inventors: Brian Mark BERG, Evansville, IN (US); Maciej Witalis CHICHLOWSKI, Evansville, IN (US); Rosaline Victoria WAWORUNTU, Evansville, IN (US)

(73) Assignee: MEAD JOHNSON NUTRITION COMPANY, Evansville, IN (US)

(21) Appl. No.: 17/052,750

(22) PCT Filed: May 9, 2019
(86) PCT No.: PCT/EP2019/061942

§ 371 Date: Nov. 3, 2020

(30) Foreign Application Priority Data
May 9, 2018 (GB) 1807539.0

(51) Int. Cl. A23C 9/20; A23L 33/00; A23L 33/125; A23L 33/135; A23C 9/13; A23C 9/127

(52) U.S. Cl. CPC A23C 9/206; A23L 33/30; A23C 9/1275; A23L 33/135; A23C 9/1307; A23L 33/125

(57) ABSTRACT

The present disclosure generally provides nutritional compositions that are useful for promoting beneficial bacteria in the gastrointestinal tract of a Cesarean-section (C-section)-delivered infant. The nutritional composition can include a prebiotic composition comprising human milk oligosaccharides (HMO), milk fat globule membrane (MFGM), and galacto-oligosaccharides (GOS) and/or polydextrose (PDX). The present disclosure also provides methods for promoting the growth of beneficial microbiota in the gastrointestinal tract of C-section-delivered infants comprising administering to a C-section-delivered infant the disclosed nutritional composition.

### TECHNICAL FIELD

The present disclosure generally provides nutritional compositions that are useful for promoting beneficial bacteria in the gastrointestinal tract of a Cesarean-section (C-section)-delivered infant. The nutritional composition can include a prebiotic composition comprising, human milk oligosaccharides (HMO), milk fat globule membrane (MFGM), and galacto-oligosaccharides (GOS) and/or polydextrose (PDX). The present disclosure also provides methods for promoting the growth of beneficial microbiota in the gastrointestinal tract of C-section-delivered infants comprising administering to a C-section-delivered infant the disclosed nutritional composition.

### BACKGROUND

Infancy is a critical stage for the foundation and development of the microbiome. The first major microbial exposure for a vaginally born infant is in the birth canal, a potentially important event for establishing a healthy microbiome early in life. C-section bypasses this exposure, altering the initial pool of microbes to which the neonate is exposed.

C-section disrupts microbiome establishment and adversely affects health later in life, e.g., an associated risk of immune-related and other diseases. (See, Sevelsted et al. (2015) PEDIATRICS 135(1):e92-e98.) In C-section infants (born without rupture of amniotic fluid) there is an absence of bacterial groups normally transmitted during vaginal delivery. For example, Lactobacillus spp. is virtually absent in the early C-section microbiota, which is dominated by skin-resident bacteria (e.g., Staphylococcus, Corynebacterium, Propionibacterium spp.). (See, Dominguez-Bello et al. (2010) PNAS 107(26):11971-11975). Unlike vaginally born babies, those born by C-section harbor no vaginal microbes (e.g., Prevotella, Sneathia spp.) at birth. (Id.)

In a study by Bokulich et al. (2017), C-section significantly altered microbial β-diversity, a measure of similarities between samples as a function of microbial composition, compared to vaginally born children. (See, Bokulich et al. (2017) SCIENCE TRANSLATIONAL MEDICINE 8(343):343ra382.) Most prominently, Bacteroidetes populations was significantly lower in C-section infants. (Id.) Also, Clostridiales and Enterobacteriaceae were significantly more abundant in C-section infants during the first year of life.

Vaginally born infants are initially colonized by fecal and vaginal bacteria from the mother, whereas infants born via C-section are colonized by bacteria from the hospital environment (e.g., health-care workers/air, equipment, other newborns). (Penders et al. (2006) Pediatrics 118(2):511-521; Biasucci et al. (2008) The Journal of Nutrition 138(9):1796S-1800S.) Newborns delivered by C-section have in general lower numbers of Bifidobacteria, reduced levels of members of the Bacteroides fragilis group and higher amounts of Clostridium difficile compared to vaginally born infants. (Penders, supra.) Moreover, the growth of Bacteroides, Bifidobacterium and Escherichia coli is delayed in infants born by C-section (Biasucci (2008), supra; Biasucci et al. (2010) Early Hum Dev. 86 Suppl. 1:13-15.)

As a consequence of alterations of gut microbiota establishment in C-section infants compared to vaginally born counterparts, study has shown that C-section birth is associated with an increased likelihood of immune and metabolic disorders such as allergies, asthma, hypertension, and obesity. In early life, the gut microbiota play significant roles in influencing the development and maturation of immune system. Thus, perturbation of early microbial environment may lead to the development of the above disorders. (Hansen et al. (2014) THE JOURNAL OF IMMUNOLOGY 193(3):1213-1222.)

C-section birth has also been related to the increase of autism spectrum disorder by 23% (Curran (2015) JOURNAL OF CHILD PSYCHOLOGY AND PSYCHIATRY, 56(5):500-508; Curran et al. (2015) JAMA PSYCHIATRY 72(9):935-942), and the risk of delay in cognitive and motor development at age 9 months. (Khalaf et al. (2015) SOCIAL PSYCHIATRY AND PSYCHIATRIC EPIDEMIOLOGY 50(10):1557-1567.) These observations are not surprising since the establishment of gut microbiota occurs at the same time with brain development and studies in animal models had demonstrated that gut microbiota is needed for myelination and normal brain development. (Hoban et al. (2016) TRANSLATIONAL PSYCHIATRY 6(4):e774.)

Accordingly, there is a need to provide nutritional compositions, such as infant formulas, that promote the growth of healthy gut microbiota, and promote a healthy gut-brain axis in a C-section infant. The present disclosure addresses this need by providing nutritional compositions comprising prebiotic, HMO and a probiotic.

### BRIEF SUMMARY

The present disclosure is directed to a nutritional composition comprising HMO, MFGM, and GOX and/or PDX. While not being bound by any particular theory, it is believed that HMO, MFGM, and GOX and/or PDX may act synergistically when included in nutritional compositions, such as infant formulas, to promote the growth and/or function of beneficial gut microbiota, thereby stimulating the gut-brain axis.

It is to be understood that both the foregoing general description and the following detailed description present aspects of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

In one aspect, the disclosure relates to a nutritional composition for an infant delivered by C-section, the nutritional composition comprising: (i) a human milk oligosaccharide or a precursor thereof, (ii) milk fat globule membrane (MFGM), and (iii) galacto-oligosaccharide (GOS) and/or polydextrose (PDX).

In another aspect, the disclosure relates to a method of promoting the growth of beneficial microbiota in the gastrointestinal tract of C-section infants, the method comprising providing to the infant a nutritional composition comprising: (i) a human milk oligosaccharide or a precursor thereof, (ii) milk fat globule membrane (MFGM), and (iii) galacto-oligosaccharide (GOS) and/or polydextrose (PDX). The method may further promote the development and stabilization of a healthy core microbiome in the C-section infant, the healthy core microbiome comprising at least one bacterial species capable of: a. transcription, translation, or energy production; b. modulating adhesion of bacteria to the C-section infant's gut epithelium; or c. producing compounds beneficial for the functioning of the C-section infant's gut. The healthy core microbiome may comprise bacterial species capable of: a. transcription, translation, or energy production; b. modulating adhesion of bacteria to the C-section infant's gut epithelium; and c. producing compounds beneficial for the functioning of the C-section infant's gut. The bacterial species may be selected from the group consisting of Faecalibacterim praustnitzii, Bifidobacterium sp., Lactobacillus sp., B. fragilis, L. reuteri, Ruminococcus sp., Clostridium cluster XIVa, Clostridium cluster IV, and Clostridium cluster VIII. The bacterial species may comprise B. longum or B. bifidum.

In another aspect, the disclosure relates to a method of modifying the ratio of Bacteroidetes to Firmicutes in a C-section infant to resemble that of a breast-fed infant, the method comprising providing to the infant a nutritional composition comprising: (i) a human milk oligosaccharide or a precursor thereof, (ii) milk fat globule membrane (MFGM), and (iii) galacto-oligosaccharide (GOS) and/or polydextrose (PDX).

In the disclosed compositions and methods, the at least one human milk oligosaccharide may comprise 2′-fucosyllactose, 3′-fucosyllactose, 3′sialyllactose, 6′sialyllactose, lacto-N-biose, lacto-N-neotetraose, lacto-N-tetraose, or any combination thereof. The human milk oligosaccharide may be present at a concentration ranging from about 0.5 mg/ml to about 10 mg/ml of the nutritional composition. The MFGM may be present in an amount of about 1.5 mg/ml to about 7.5 mg/ml of the nutritional composition. The GOS and/or PDX may be present in an amount of about 1 mg/ml to about 6 mg/ml.

### BRIEF DESCRIPTION OF THE DRAWINGS

This patent application contains at least one drawing executed in color. Copies of this patent application with color drawing(s) will be provided by the Office upon request and payment of the necessary fee.

FIG. 1 illustrates the principle components plot (PCoA) of mouse fecal microbiota community structure (β-diversity; Unweighted UniFrac) comparing diet conditions. Microbiome of mice receiving GOS+PDX+sialyllactose (“SAL”) clusters separately versus Control-fed mice or SAL-fed mice (ADONIS p<0.05).

FIG. 2A illustrates heat maps of fecal metabolites differentially altered in A) GOS+SAL+PDX-fed mice compared to Control, chow-fed mice or B) SAL-fed mice compared to Control, chow-fed mice. All selected metabolites were confirmed as significant by Random Forest and Boruta feature selection when applied to all three groups simultaneously.

FIG. 2B illustrates heat maps of fecal metabolites differentially altered in A) GOS+SAL+PDX-fed mice compared to Control, chow-fed mice or B) SAL-fed mice compared to Control, chow-fed mice. All selected metabolites were confirmed as significant by Random Forest and Boruta feature selection when applied to all three groups simultaneously.

### DETAILED DESCRIPTION

Reference now will be made in detail to the aspects of the present disclosure, one or more examples of which are set forth herein below. Each example is provided by way of explanation of the nutritional composition of the present disclosure and is not a limitation. In fact, it will be apparent to those skilled in the art that various modifications and variations can be made to the teachings of the present disclosure without departing from the scope or spirit of the disclosure. For instance, features illustrated or described as part of one aspect, can be used with another aspect to yield a still further aspect.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary aspects only and is not intended as limiting the broader aspects of the present disclosure.

“Nutritional composition” means a substance or formulation that satisfies at least a portion of a subject's nutrient requirements. The terms “nutritional(s),” “nutritional formula(s),” “enteral nutritional(s),” “nutritional composition(s),” and “nutritional supplement(s)” are used interchangeably throughout the present disclosure to refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults, such as women who are lactating or pregnant. The nutritional compositions may be for pediatric subjects, including infants and children.

The term “synthetic” when applied to a composition, nutritional composition, or mixture means a composition, nutritional composition, or mixture obtained by biological and/or chemical means, which can be chemically identical to the mixture naturally occurring in mammalian milks. A composition, nutritional composition, or mixture is said to be “synthetic” if at least one of its components is obtained by biological (e.g. enzymatic) and/or chemical means.

The term “enteral” means through or within the gastrointestinal, or digestive, tract. “Enteral administration” includes oral feeding, intragastric feeding, transpyloric administration, or any other administration into the digestive tract.

“Pediatric subject” includes both infants and children, and refers herein to a human that is less than thirteen years of age. A pediatric subject may refer to a human subject that is less than eight years old. A pediatric subject may refer to a human subject between about one and about six years of age or about one and about three years of age. A pediatric subject may refer to a human subject between about 6 and about 12 years of age.

“Infant” means a subject having an age of not more than about one year and includes infants from about zero to about twelve months. The term infant includes low birth weight infants, very low birth weight infants, and preterm infants. “Preterm” means an infant born before the end of the 37th week of gestation, while “full term” means an infant born after the end of the 37th week of gestation.

“Child” means a subject ranging in age from about twelve months to about thirteen years. A child may be a subject between the ages of one and twelve years old. The terms “children” or “child” may refer to subjects that are between about one and about six years old, between about one and about three years old, or between about seven and about twelve years old. The terms “children” or “child” may refer to any range of ages between about 12 months and about 13 years.

“Children's nutritional product” refers to a composition that satisfies at least a portion of the nutrient requirements of a child. A growing-up milk is an example of a children's nutritional product.

“Infant formula” means a composition that satisfies at least a portion of the nutrient requirements of an infant. In the United States, the content of an infant formula is dictated by the federal regulations set forth at 21 C.F.R. Sections 100, 106, and 107. These regulations define macronutrient, vitamin, mineral, and other ingredient levels in an effort to simulate the nutritional and other properties of human breast milk.

“Milk fat globule membrane” (“MFGM”) includes components found in the milk fat globule membrane including but not limited to milk fat globule membrane proteins such as Mucin 1, Butyrophilin, Adipophilin, CD36, CD14, Lactadherin (PAS6/7), Xanthine oxidase and Fatty Acid binding proteins etc. Additionally, “milk fat globule membrane” may include phospholipids, cerebrosides, gangliosides, sphingoids or sphingolipids, and/or cholesterol.

The term “growing-up milk” refers to a broad category of nutritional compositions intended to be used as a part of a diverse diet in order to support the normal growth and development of a child between the ages of about 1 and about 6 years of age.

“Milk-based” means comprising at least one component that has been drawn or extracted from the mammary gland of a mammal. A milk-based nutritional composition may comprise components of milk that are derived from domesticated ungulates, ruminants or other mammals or any combination thereof. Moreover, milk-based may mean comprising bovine casein, whey, lactose, or any combination thereof. Further, “milk-based nutritional composition” may refer to any composition comprising any milk-derived or milk-based product known in the art.

“Nutritionally complete” means a composition that may be used as the sole source of nutrition, which would supply essentially all of the required daily amounts of vitamins, minerals, and/or trace elements in combination with proteins, carbohydrates, and lipids. Indeed, “nutritionally complete” describes a nutritional composition that provides adequate amounts of carbohydrates, lipids, essential fatty acids, proteins, essential amino acids, conditionally essential amino acids, vitamins, minerals and energy required to support normal growth and development of a subject.

Therefore, a nutritional composition that is “nutritionally complete” for a preterm infant will, by definition, provide qualitatively and quantitatively adequate amounts of carbohydrates, lipids, essential fatty acids, proteins, essential amino acids, conditionally essential amino acids, vitamins, minerals, and energy required for growth of the preterm infant.

A nutritional composition that is “nutritionally complete” for a term infant will, by definition, provide qualitatively and quantitatively adequate amounts of all carbohydrates, lipids, essential fatty acids, proteins, essential amino acids, conditionally essential amino acids, vitamins, minerals, and energy required for growth of the term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

As applied to nutrients, the term “essential” refers to any nutrient that cannot be synthesized by the body in amounts sufficient for normal growth and to maintain health and that, therefore, must be supplied by the diet. The term “conditionally essential” as applied to nutrients means that the nutrient must be supplied by the diet under conditions when adequate amounts of the precursor compound is unavailable to the body for endogenous synthesis to occur.

“Nutritional supplement” or “supplement” refers to a formulation that contains a nutritionally relevant amount of at least one nutrient. For example, supplements described herein may provide at least one nutrient for a human subject, such as a lactating or pregnant female.

“Probiotic” means a microorganism with low or no pathogenicity that exerts at least one beneficial effect on the health of the host. An example of a probiotic is LGG.

The probiotic(s) may be viable or non-viable. As used herein, the term “viable”, refers to live microorganisms. The term “non-viable” or “non-viable probiotic” means non-living probiotic microorganisms, their cellular components and/or metabolites thereof. Such non-viable probiotics may have been heat-killed or otherwise inactivated, but they retain the ability to favorably influence the health of the host. The probiotics useful in the present disclosure may be naturally-occurring, synthetic or developed through the genetic manipulation of organisms, whether such source is now known or later developed.

The term “non-viable probiotic” means a probiotic wherein the metabolic activity or reproductive ability of the referenced probiotic has been reduced or destroyed. More specifically, “non-viable” or “non-viable probiotic” means non-living probiotic microorganisms, their cellular components and/or metabolites thereof. Such non-viable probiotics may have been heat-killed or otherwise inactivated. The “non-viable probiotic” does, however, still retain, at the cellular level, its cell structure or other structure associated with the cell, for example exopolysaccharide and at least a portion its biological glycol-protein and DNA/RNA structure and thus retains the ability to favorably influence the health of the host. Contrariwise, the term “viable” refers to live microorganisms. As used herein, the term “non-viable” is synonymous with “inactivated”.

The term “cell equivalent” refers to the level of non-viable, non-replicating probiotics equivalent to an equal number of viable cells. The term “non-replicating” is to be understood as the amount of non-replicating microorganisms obtained from the same amount of replicating bacteria (cfu/g), including inactivated probiotics, fragments of DNA, cell wall or cytoplasmic compounds. In other words, the quantity of non-living, non-replicating organisms is expressed in terms of cfu as if all the microorganisms were alive, regardless whether they are dead, non-replicating, inactivated, fragmented etc.

“Prebiotic” means a non-digestible food ingredient that beneficially affects the host by selectively stimulating the growth and/or activity of one or a limited number of beneficial gut bacteria in the digestive tract, selective reduction in gut pathogens, or favorable influence on gut short chain fatty acid profile that can improve the health of the host.

“β-glucan” means all β-glucan, including both β-1,3-glucan and β-1,3;1,6-glucan, as each is a specific type of β-glucan. Moreover, β-1,3;1,6-glucan is a type of β-1,3-glucan. Therefore, the term “β-1,3-glucan” includes β-1,3;1,6-glucan.

All percentages, parts and ratios as used herein are by weight of the total formulation, unless otherwise specified.

The nutritional composition of the present disclosure may be free of substantially free of any optional or selected ingredients described herein. In this context, and unless otherwise specified, the term “substantially free” means that the selected composition may contain less than a functional amount of the optional ingredient, typically less than 0.1% by weight, and also, including zero percent by weight of such optional or selected ingredient.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The compositions and methods of the present disclosure, including components thereof, can comprise, consist of, or consist essentially of the essential elements and limitations of the aspects described herein, as well as any additional or optional ingredients, components or limitations described herein or otherwise useful in nutritional compositions.

As used herein, the term “about” should be construed to refer to both of the numbers specified in any range. Any reference to a range should be considered as providing support for any subset within that range.

The present disclosure generally relates to compositions and methods for promoting the growth of beneficial microbiota in the gastrointestinal tract of C-section infants. It is well documented that infants delivered by C-section are at increased risk for difficulties with breast feeding. Accordingly, the present disclosure provides compositions to meet the increased need of C-section infants for pediatric nutrition support.

Overall, presence of the bacterial taxa belonging to Bacteroides, Parabacteroides, Clostridium, Lactobacillus, Bifidobacterium, and bacterial species Faecalibacterium prausnitzii is a determinant of a healthy infant microbiome. These bacterial taxa are the main producers of short chain fatty acids (SCFA), an important source of energy from nondigestible carbohydrates. (Byrne et al. (2015) INTERNATIONAL JOURNAL OF OBESITY (2005), 3 (9): 1331-1338. SCFA are also immunomodulatory (Smith et al. (2013) Science, 341(6145):569-573) and inhibit common pathogens. However, C-section is associated with enrichment for opportunistic pathogens, e.g. Haemophilus spp., Enterobacter taylorae, Veillonella dispar (Bäckhed et al. (2015) CELL HOST & MICROBE 17(6):852) and Staphylococcus (Dominguez-Bello et al. (2010) PROC NATL ACAD SCI USA 107(26):11971-11975). These microbes continue to persist at least throughout the first year of life (Bäckhed, supra) and possibly contribute to infant infection burden.

The compositions described herein may be used in a method of modulating the development and stabilization of a healthy core microbiome of C-section infants. The development and stabilization of a healthy core microbiome modulates metabolic and other molecular functions as described in more detail below.

The nutritional composition may promote the development of a healthy core microbiome which includes at least three bacterial groups associated with:

(1) housekeeping functions, such as transcription and translation, energy production (e.g., Faecalibacterim praustnitzii, Bifidobacterium sp. (e.g. B. longum, B. bifidum), Lactobacillus sp.);

(2) processes that are specific to adhesion to host cell surfaces (e.g. gut epithelium) and the production of compounds important in host-microbe interaction (including essential vitamins, such as vitamin K, and immunostimulatory compounds) (e.g. B. fragilis, L. reuteri); and

(3) gut core functions including glycosaminoglycan biodegradation, the production of several short-chain fatty acids (SCFA), enrichment for specific lipopolysaccharides, and the production of vitamins and essential amino acids (e.g. Ruminococcus sp., Clostridium cluster XIVa, IV, and VIII).

The compositions described herein promote normal microbiome development and production of beneficial microbial products. The nutritional compositions and methods herein may minimize or eliminate the differences observed in the gut microbiome between infants delivered vaginally and by C-section. The compositions and methods described herein may promote a microbiome which is less characteristic of an adult microbiota (e.g. bile acid synthesis, methanogenesis, and the phosphotransferase system) and more characteristic of a breast fed infants (e.g. synthesis of B vitamins and oxidative phosphorylation). Further, the compositions and methods described herein can modify the ratio of Bacteroidetes to Firmicutes in a C-section infant to resemble that of a breast-fed infant. The compositions and methods described herein may increase Foxp3+ regulatory T-cell through enrichment of Bifidobacterium sp. from prebiotic activities of human milk oligosaccharides.

When administered to a C-section infant, the nutritional compositions described herein may (1) normalize the gut microbiota composition in C-section infant by increasing the levels of beneficial bacterial species; (2) promote a healthy microbiome core by increasing levels of certain bacteria from the phylum Bacteroidetes, such as Bacteroides fragilis, Parabacteroides, increasing Lactobacillus species (e.g. L. reuteri), increasing certain Bifidobacteria species); (3) suppress growth of pathogenic bacteria, for example Hemophilus spp., Enterobacter taylorae, E. hormaechei, Veillonella dispar, and Staphylococcus which are opportunistic in C-section infants, (4) maintain a ratio of Firmicutes to Bacteroidetes at the level of vaginally-delivered infants (about 0.4); (5) modulate the microbial production of short chain fatty acids (SCFA) produced by beneficial bacteria, such as butyrate, propionate and acetate; (6) support development of healthy immune response by modulating regulatory immune system; (7) support development of cognitive functions through microbiota-gut-brain axis pathway. While not being bound by theory, it is believed that the interactions across the developing gut-brain axis promote neurological development and function in pediatric populations.

Accordingly, the present disclosure provides a nutritional composition comprising: (i) a human milk oligosaccharide or a precursor thereof; and (ii) galacto-oligosaccharide (GOS) and/or polydextrose (PDX). The present disclosure also provides a nutritional composition comprising: (i) a human milk oligosaccharide or a precursor thereof; (ii) milk fat globule membrane (MFGM); and (iii) galacto-oligosaccharide (GOS) and/or polydextrose (PDX). Further, the present disclosure provides a nutritional composition comprising: (i) a protein source, (ii) a lipid source, (iii) a carbohydrate source, (iv) a human milk oligosaccharide or a precursor thereof, (v) MFGM, (vi) a prebiotic comprising GOS and/or PDX. The nutritional composition may be derived from non-human milk sources, such as bovine milk, porcine milk, equine milk, buffalo milk, goat milk, murine milk, or camel milk. Alternatively, the nutritional composition may be a synthetic nutritional composition.

The term “HMO” or “human milk oligosaccharides” refers generally to a number of complex carbohydrates found in human breast milk that can be in either acidic or neutral form. HMO are generally composed of five monosaccharides: glucose, galactose, GlcNAc, L-fucose and sialic acid. The HMO may be 2′-fucosyllactose, 3′-fucosyllactose, 3′sialyllactose, 6′sialyllactose, lacto-N-biose, lacto-N-neotetraose, lacto-N-tetraose, or any combination thereof. 3′sialyllactose, 6′sialyllactose contribute sialic acid, which is an important nutrient for brain development and cognitive function. HMO may be isolated or enriched from milk or produced by microbial fermentation, enzymatic processes, chemical synthesis, or a combination thereof. Exemplary HMO precursors include sialic acid, fucose, or a combination thereof.

HMO are believed to correlate with the presence of beneficial infant specific Bifidobacterium species, such as B. longum, B. infantis, B. breve, and B. bifidium in breast fed infants. Accordingly, the HMO used in the present compositions may provide infant formulas that are functionally closer to human milk. The HMO may be present in the compositions in an amount ranging from about 0.005 g/100 kcal to about 1 g/100 kcal. The HMO may be present in an amount ranging from about 0.01 g/100 kcal to about 0.1 g/100 kcal, about 0.015 g/100 kcal to about 0.05 g/100 kcal.

The nutritional composition may include MFGM. The MFGM may be present in an amount of about 1.5 mg/ml to about 7.5 mg/ml of the nutritional composition.

MFGM may be supplied by the inclusion of an enriched milk product, such as an enriched whey protein concentrate (eWPC) in the nutritional composition. Enriched milk product generally refers to a milk product that has been enriched with certain milk fat globule membrane (MFGM) components, such as proteins and lipids found in the MFGM. The enriched milk product can be formed by, e.g., fractionation of non-human (e.g., bovine) milk. Enriched milk products have a total protein level which can range between 20% and 90%, more preferably between 68% and 80%, of which between 3% and 50% is MFGM proteins; MFGM proteins may make up from 7% to 13% of the enriched milk product protein content. Enriched milk products also comprise from 0.5% to 5% (and, at times, 1.2% to 2.8%) sialic acid, from 2% to 25% (and, in some aspects, 4% to 10%) phospholipids, from 0.4% to 3% sphingomyelin, from 0.05% to 1.8%, and, in certain aspects 0.10% to 0.3%, gangliosides and from 0.02% to about 1.2%, more preferably from 0.2% to 0.9%, cholesterol. Thus, enriched milk products include desirable components at levels higher than found in bovine and other non-human milks.

The enriched milk product may contain certain polar lipids such as (1) Glycerophospholipids such as phosphatidylcholine (PC), phosphatidylethanolamine (PE), phosphatidylserine (PS), and phosphatidylinositol (PI), and their derivatives and (2) Sphingoids or sphingolipids such as sphingomyelin (SM) and glycosphingolipids comprising cerebrosides (neutral glycosphingolipids containing uncharged sugars) and the gangliosides (acidic glycosphingolipids containing sialic acid) and their derivatives.

PE is a phospholipid found in biological membranes, particularly in nervous tissue such as the white matter of brain, nerves, neural tissue, and in spinal cord, where it makes up 45% of all phospholipids. Sphingomyelin is a type of sphingolipid found in animal cell membranes, especially in the membranous myelin sheath that surrounds some nerve cell axons. It usually consists of phosphocholine and ceramide, or a phosphoethanolamine head group; therefore, sphingomyelins can also be classified as sphingophospholipids. In humans, SM represents ^{˜}85% of all sphingolipids, and typically makes up 10-20 mol % of plasma membrane lipids. Sphingomyelins are present in the plasma membranes of animal cells and are especially prominent in myelin, a membranous sheath that surrounds and insulates the axons of some neurons.

The enriched milk product may include eWPC. The eWPC may be produced by any number of fractionation techniques. These techniques include but are not limited to melting point fractionation, organic solvent fractionation, super critical fluid fractionation, and any variants and combinations thereof. Alternatively, eWPC is available commercially, including under the trade names Lacprodan MFGM-10 and Lacprodan PL-20, both available from Aria Food Ingredients of Viby, Denmark. With the addition of eWPC, the lipid composition of infant formulas and other pediatric nutritional compositions can more closely resemble that of human milk. For instance, the theoretical values of phospholipids (mg/L) and gangliosides (mg/L) in an exemplary infant formula which includes Lacprodan MFGM-10 or Lacprodan PL-20 can be calculated as shown in Table 1:

TABLE 1

| | Total | | | | | | Other | |
|---|---|---|---|---|---|---|---|---|
| Item | milk PL | SM | PE | PC | PI | PS | PL | GD3 |
| MFGM-10 | 330 | 79.2 | 83.6 | 83.6 | 22 | 39.6 | 22 | 10.1 |
| PL-20 | 304 | 79 | 64 | 82 | 33 | 33 | 12.2 | 8.5 |

PL: phospholipids;
SM: sphingomyelin;
PE: phosphatidyl ethanolamine;
PC: phosphatidyl choline;
PI: phosphatidyl inositol;
PS: phosphatidyl serine;
GD3: ganglioside GD3.

The eWPC may be included in the nutritional composition at a level of about 0.5 grams per liter (g/L) to about 10 g/L; the eWPC may be present at a level of about 1 g/L to about 9 g/L. The eWPC may be present in the nutritional composition at a level of about 3 g/L to about 8 g/L. Alternatively, the eWPC may be included in the preterm nutritional composition of the present disclosure at a level of about 0.06 grams per 100 Kcal (g/100 Kcal) to about 1.5 g/100 Kcal; the eWPC may be present at a level of about 0.3 g/100 Kcal to about 1.4 g/100 Kcal. The eWPC may be present in the nutritional composition at a level of about 0.4 g/100 Kcal to about 1 g/100 Kcal.

Total phospholipids in the nutritional compositions disclosed herein (i.e., including phospholipids from the eWPC as well as other components, but not including phospholipids from plant sources such as soy lecithin, if used) is in a range of about 50 mg/L to about 2000 mg/L; it may be about 100 mg/L to about 1000 mg/L, or about 150 mg/L to about 550 mg/L. The eWPC component may also contribute sphingomyelin in a range of about 10 mg/L to about 200 mg/L; it may be about 30 mg/L to about 150 mg/L, or about 50 mg/L to about 140 mg/L. And, the eWPC can also contribute gangliosides, which may be present in a range of about 2 mg/L to about 40 mg/L, or about 6 mg/L to about 35 mg/L. The gangliosides may be present in a range of about 9 mg/L to about 30 mg/L. Total phospholipids in the nutritional composition (again not including phospholipids from plant sources such as soy lecithin) may be in a range of about 6 mg/100 Kcal to about 300 mg/100 Kcal; it may be about 12 mg/100 Kcal to about 150 mg/100 Kcal, or about 18 mg/100 Kcal to about 85 mg/100 Kcal. The eWPC may also contribute sphingomyelin in a range of about 1 mg/100 Kcal to about 30 mg/100 Kcal; it may be about 3.5 mg/100 Kcal to about 24 mg/100 Kcal, or about 6 mg/100 Kcal to about 21 mg/100 Kcal. And, gangliosides may be present in a range of about 0.25 mg/100 Kcal to about 6 mg/100 Kcal, or about 0.7 mg/100 Kcal to about 5.2 mg/100 Kcal. The gangliosides may be present in a range of about 1.1 mg/100 Kcal to about 4.5 mg/100 Kcal.

The eWPC may contain sialic acid (SA). Generally, the term sialic acid (SA) is used to generally refer to a family of derivatives of neuraminic acid. N-acetylneuraminic acid (Neu5Ac) and N-glycolylneuraminic acid (Neu5Gc) are among the most abundant naturally found forms of SA, especially Neu5Ac in human and cow's milk. Mammalian brain tissue contains the highest levels of SA because of its incorporation into brain-specific proteins such as neural cell adhesion molecule (NCAM) and lipids (e.g., gangliosides). It is considered that SA plays a role in neural development and function, learning, cognition, and memory throughout the life. In human milk, SA exists as free and bound forms with oligosaccharides, protein and lipid. The content of SA in human milk varies with lactation stage, with the highest level found in colostrum. However, most SA in bovine milk is bound with proteins, compared to the majority of SA in human milk bound to free oligosaccharides. Sialic acid can be incorporated in to the disclosed preterm infant formula as is, or it can be provided by incorporating casein glycomacropeptide (cGMP) having enhanced sialic acid content, as discussed in U.S. Pat. Nos. 7,867,541 and 7,951,410, the disclosure of each of which are incorporated by reference herein.

When present, sialic acid may be incorporated into the nutritional composition of the present disclosure at a level of about 100 mg/L to about 800 mg/L, including both inherent sialic acid from the eWPC and exogenous sialic acid and sialic acid from sources such as cGMP. Sialic acid may be present at a level of about 120 mg/L to about 600 mg/L; the level may be about 140 mg/L to about 500 mg/L. Sialic acid may be present in an amount from about 1 mg/100 Kcals to about 120 mg/100 Kcal. Sialic acid may be present in an amount from about 14 mg/100 Kcal to about 90 mg/100 Kcal. Sialic acid may be present in an amount from about 15 mg/100 Kcal to about 75 mg/100 Kcal.

The disclosed nutritional composition also comprises a source of prebiotics, specifically GOS and/or PDX. At least 20% of the prebiotics may comprise GOS. The prebiotic component may comprise both GOS and PDX. The GOS and PDX may be present in a ratio of about 1:9 to about 9:1 by weight. The GOS and PDX may be present in a ratio of about 1:4 to 4:1, or about 1:1.

The amount of GOS in the nutritional composition may be from about 0.1 g/100 kcal to about 1.0 g/100 kcal. The amount of GOS in the nutritional composition may be from about 0.1 g/100 kcal to about 0.5 g/100 kcal. The amount of PDX in the nutritional composition may be within the range of from about 0.1 g/100 kcal to about 0.5 g/100 kcal. The amount of PDX may be about 0.3 g/100 kcal.

GOS and PDX may be supplemented into the nutritional composition in a total amount of about at least about 0.2 g/100 kcal and can be about 0.2 g/100 kcal to about 1.5 g/100 kcal. The nutritional composition may comprise GOS and PDX in a total amount of from about 0.6 to about 0.8 g/100 kcal.

The nutritional composition may comprise Lactobacillus rhamnosus GG (ATCC number 53103). Other probiotics useful in the present nutritional compositions include, but are not limited to, Bifidobacterium species such as Bifidobacterium longum BB536 (BL999, ATCC: BAA-999), and Bifidobacterium animalis subsp. lactis BB-12 (DSM No. 10140) or any combination thereof.

LGG and prebiotics, such as GOS and PDX, are believed to significantly and surprisingly improve brain development, cognitive function, and even social and emotional skills. Additionally, the administration of a combination of GOS, PDX and LGG may alter the production of neurotransmitters, such as serotonin, 5-hydroxytryptophan, noradrenaline and/or 5-hydroxyindoleacetic acid. The ability of the compositions to modulate neurotransmitters may explain the beneficial effects of the present compositions on social skills, anxiety and memory function.

The nutritional composition may include a probiotic, and more particularly, LGG, in an amount of from about 1×10⁴ cfu/100 kcal to about 1.5×10¹⁰ cfu/100 kcal. The nutritional composition may comprise LGG in an amount of from about 1×10⁶ cfu/100 kcal to about 1×10⁹ cfu/100 kcal. Still, the nutritional composition may include LGG in an amount of from about 1×10⁷ cfu/100 kcal to about 1×10⁸ cfu/100 kcal. Where LGG is not included at the upper limit of the concentration range, additional probiotics may be included up to the upper limit concentration specified. The probiotic may be either non-viable or viable.

The probiotic functionality in the nutritional composition of the present disclosure may be provided by including a culture supernatant from a late-exponential growth phase of a probiotic batch-cultivation process, as disclosed in international published application no. WO 2013/142403, which is hereby incorporated by reference in its entirety. Without wishing to be bound by theory, it is believed that the activity of the culture supernatant can be attributed to the mixture of components (including proteinaceous materials, and possibly including (exo)polysaccharide materials) as found released into the culture medium at a late stage of the exponential (or “log”) phase of batch cultivation of the probiotic. The term “culture supernatant” as used herein, includes the mixture of components found in the culture medium. The stages recognized in batch cultivation of bacteria are known to the skilled person. These are the “lag,” the “log” (“logarithmic” or “exponential”), the “stationary” and the “death” (or “logarithmic decline”) phases. In all phases during which live bacteria are present, the bacteria metabolize nutrients from the media, and secrete (exert, release) materials into the culture medium. The composition of the secreted material at a given point in time of the growth stages is not generally predictable.

A culture supernatant may be obtainable by a process comprising the steps of (a) subjecting a probiotic such as LGG to cultivation in a suitable culture medium using a batch process; (b) harvesting the culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5-6 kiloDaltons (kDa); (d) removing liquid contents from the culture supernatant so as to obtain the composition.

The culture supernatant may comprise secreted materials that are harvested from a late exponential phase. The late exponential phase occurs in time after the mid exponential phase (which is halftime of the duration of the exponential phase, hence the reference to the late exponential phase as being the second half of the time between the lag phase and the stationary phase). In particular, the term “late exponential phase” is used herein with reference to the latter quarter portion of the time between the lag phase and the stationary phase of the LGG batch-cultivation process. The culture supernatant may be harvested at a point in time of 75% to 85% of the duration of the exponential phase, and may be harvested at about ⅚ of the time elapsed in the exponential phase.

The nutritional composition of the disclosure may contain a source of long chain polyunsaturated fatty acid (LCPUFA) that comprises docosahexaenoic acid. Other suitable LCPUFAs include, but are not limited to, α-linoleic acid, γ-linoleic acid, linoleic acid, linolenic acid, docosahexaenoic acid (DHA), eicosapentaenoic acid (EPA) and arachidonic acid (ARA).

Especially if the nutritional composition is an infant formula, the nutritional composition may be supplemented with both DHA and ARA. The weight ratio of ARA:DHA may be between about 1:3 and about 9:1. The ratio of ARA:DHA may be from about 1:2 to about 4:1.

If included, the source of DHA and/or ARA may be any source known in the art such as marine oil, fish oil, single cell oil, egg yolk lipid, and brain lipid. The DHA and ARA may be sourced from single cell Martek oils, DHASCO® and ARASCO®, or variations thereof. The DHA and ARA can be in natural form, provided that the remainder of the LCPUFA source does not result in any substantial deleterious effect on the subject. Alternatively, the DHA and ARA can be used in refined form.

Sources of DHA and ARA may be single cell oils as taught in U.S. Pat. Nos. 5,374,657; 5,550,156; and 5,397,591, the disclosures of which are incorporated herein in their entirety by reference. Nevertheless, the present disclosure is not limited to only such oils.

The nutritional composition may also comprise a source of β-glucan. Glucans are polysaccharides, specifically polymers of glucose, which are naturally occurring and may be found in cell walls of bacteria, yeast, fungi, and plants. Beta glucans (β-glucans) are themselves a diverse subset of glucose polymers, which are made up of chains of glucose monomers linked together via beta-type glycosidic bonds to form complex carbohydrates.

β-1,3-glucans are carbohydrate polymers purified from, for example, yeast, mushroom, bacteria, algae, or cereals. (Stone B A, Clarke A E. Chemistry and Biology of (1-3)-Beta-Glucans. London:Portland Press Ltd; 1993.) The chemical structure of β-1,3-glucan depends on the source of the β-1,3-glucan. Moreover, various physiochemical parameters, such as solubility, primary structure, molecular weight, and branching, play a role in biological activities of β-1,3-glucans. (Yadomae T., Structure and biological activities of fungal beta-1,3-glucans. Yakugaku Zasshi. 2000; 120:413-431.)

β-1,3-glucans are naturally occurring polysaccharides, with or without β-1,6-glucose side chains that are found in the cell walls of a variety of plants, yeasts, fungi and bacteria. β-1,3;1,6-glucans are those containing glucose units with (1,3) links having side chains attached at the (1,6) position(s). β-1,3;1,6 glucans are a heterogeneous group of glucose polymers that share structural commonalities, including a backbone of straight chain glucose units linked by a β-1,3 bond with β-1,6-linked glucose branches extending from this backbone. While this is the basic structure for the presently described class of β-glucans, some variations may exist. For example, certain yeast β-glucans have additional regions of β(1,3) branching extending from the β(1,6) branches, which add further complexity to their respective structures.

β-glucans derived from baker's yeast, Saccharomyces cerevisiae, are made up of chains of D-glucose molecules connected at the 1 and 3 positions, having side chains of glucose attached at the 1 and 6 positions. Yeast-derived β-glucan is an insoluble, fiber-like, complex sugar having the general structure of a linear chain of glucose units with a β-1,3 backbone interspersed with β-1,6 side chains that are generally 6-8 glucose units in length. More specifically, β-glucan derived from baker's yeast is poly-(1,6)-β-D-glucopyranosyl-(1,3)-β-D-glucopyranose.

Furthermore, β-glucans are well tolerated and do not produce or cause excess gas, abdominal distension, bloating or diarrhea in pediatric subjects. Addition of β-glucan to a nutritional composition for a pediatric subject, such as an infant formula, a growing-up milk or another children's nutritional product, will improve the subject's immune response by increasing resistance against invading pathogens and therefore maintaining or improving overall health.

A nutritional composition may comprise per 100 kcal: (i) between about 1 g and about 7 g of a protein source, (ii) between about 1 g and about 10 g of a lipid source, (iii) between about 6 g and about 22 g of a carbohydrate source, (iv) between about 0.005 g and about 1 g of a human milk oligosaccharide, (v) between about 0.1 mg and 1.0 mg of a galacto-oligosaccharide, (vi) between about 0.1 mg and about 0.5 mg of polydextrose, and (vii) between about 1×10⁵ cfu/100 kcals to about 1.5×10⁹ cfu/100 kcals of Lactobacillus rhamnosus GG or about 1×10⁵ equivalent cfu/100 kcals to about 1.5×10⁹ equivalent cfu/100 kcals of dry composition of Lactobacillus rhamnosus GG. The nutritional composition may comprise the culture supernatant from about 0.015 g per 100 kcal to about 1.5 g per 100 kcal.

The disclosed nutritional composition(s) may be provided in any form known in the art, such as a powder, a gel, a suspension, a paste, a solid, a liquid, a liquid concentrate, a reconstitutable powdered milk substitute or a ready-to-use product. The nutritional composition may comprise a nutritional supplement, children's nutritional product, infant formula, human milk fortifier, growing-up milk or any other nutritional composition designed for a pediatric subject. Nutritional compositions of the present disclosure include, for example, orally-ingestible, health-promoting substances including, for example, foods, beverages, tablets, capsules and powders. Moreover, the nutritional composition of the present disclosure may be standardized to a specific caloric content, it may be provided as a ready-to-use product, or it may be provided in a concentrated form. The nutritional composition may be in powder form with a particle size in the range of 5 μm to 1500 μm, more preferably in the range of 10 μm to 1000 μm, and even more preferably in the range of 50 μm to 300 μm.

The nutritional composition may be an infant formula suitable for infants ranging in age from 0 to 12 months, from 0 to 3 months, 0 to 6 months or 6 to 12 months. The disclosure may provide a fortified milk-based growing-up milk designed for children ages 1-3 years and/or 4-6 years, wherein the growing-up milk supports growth and development and life-long health.

Where the nutritional composition is an infant formula, the combination of HMOs, MFGM, and GOS and/or PDX may be added to a commercially available infant formula. For example, Enfalac, Enfamil®, Enfamil® Premature Formula, Enfamil® with Iron, Enfamil® Lactofree®, Nutramigen®, Pregestimil®, and ProSobee® (available from Mead Johnson & Company, Evansville, Ind., U.S.A.) may be supplemented with HMOs, MFGM, and GOS and/or PDX, and used in practice of the current disclosure.

As noted, the nutritional composition(s) of the disclosure may comprise a protein source. The protein source can be any used in the art, e.g., nonfat milk, whey protein, casein, soy protein, hydrolyzed protein, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate) and any combinations thereof.

The proteins of the nutritional composition may be provided as intact proteins. The proteins may be provided as a combination of both intact proteins and partially hydrolyzed proteins, with a degree of hydrolysis of between about 4% and 10%. The proteins may be more completely hydrolyzed. The protein source may comprise amino acids as a protein equivalent. The protein source may be supplemented with glutamine-containing peptides.

In the nutritional composition, the whey:casein ratio of the protein source may be similar to that found in human breast milk. The protein source may comprise from about 40% to about 90% whey protein and from about 10% to about 60% casein.

The nutritional composition may comprise between about 1 g and about 7 g of a protein source per 100 kcal. The nutritional composition may comprise between about 3.5 g and about 4.5 g of protein per 100 kcal.

Suitable fat or lipid sources for the nutritional composition of the present disclosure may be any known or used in the art, including but not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

Carbohydrate sources can be any used in the art, e.g., lactose, glucose, fructose, corn syrup solids, maltodextrins, sucrose, starch, rice syrup solids, and the like. The amount of carbohydrate in the nutritional composition typically can vary from between about 5 g and about 25 g/100 kcal.

The nutritional composition may include prebiotics in addition to GOS and PDX. Additional prebiotics useful in the present disclosure may include: lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, and gentio-oligosaccharides. Where GOS and PDX are not included at the upper limit of their respective concentration range, additional prebiotics may be included up to the upper limit concentration specified.

The nutritional composition of the present disclosure may comprise lactoferrin. Lactoferrins are single chain polypeptides of about 80 kDa containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe3+) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (^{˜}pl 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms.

Lactoferrin for use in the present disclosure may be, for example, isolated from the milk of a non-human animal or produced by a genetically modified organism. The oral electrolyte solutions described herein may comprise non-human lactoferrin, non-human lactoferrin produced by a genetically modified organism and/or human lactoferrin produced by a genetically modified organism.

Suitable non-human lactoferrins for use in the present disclosure include, but are not limited to, those having at least 48% homology with the amino acid sequence of human lactoferrin. For instance, bovine lactoferrin (“bLF”) has an amino acid composition which has about 70% sequence homology to that of human lactoferrin. The non-human lactoferrin may have at least 65% homology with human lactoferrin and in some aspects, at least 75% homology. Non-human lactoferrins acceptable for use in the present disclosure include, without limitation, bLF, porcine lactoferrin, equine lactoferrin, buffalo lactoferrin, goat lactoferrin, murine lactoferrin and camel lactoferrin.

The nutritional composition of the present disclosure may comprise non-human lactoferrin, for example bLF. bLF is a glycoprotein that belongs to the iron transporter or transferring family. It is isolated from bovine milk, wherein it is found as a component of whey. There are known differences between the amino acid sequence, glycosylation patters and iron-binding capacity in human lactoferrin and bLF. Additionally, there are multiple and sequential processing steps involved in the isolation of bLF from cow's milk that affect the physiochemical properties of the resulting bLF preparation. Human lactoferrin and bLF are also reported to have differences in their abilities to bind the lactoferrin receptor found in the human intestine.

Though not wishing to be bound by this or any other theory, it is believed that bLF that has been isolated from whole milk has less lipopolysaccharide (LPS) initially bound than does bLF that has been isolated from milk powder. Additionally, it is believed that bLF with a low somatic cell count has less initially-bound LPS. A bLF with less initially-bound LPS has more binding sites available on its surface. This is thought to aid bLF in binding to the appropriate location and disrupting the infection process

bLF suitable for the present disclosure may be produced by any method known in the art. For example, in U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

Lactoferrin utilized in the present disclosure may be provided by an expanded bed absorption (“EBA”) process for isolating proteins from milk sources. EBA, also sometimes called stabilized fluid bed adsorption, is a process for isolating a milk protein, such as lactoferrin, from a milk source comprises establishing an expanded bed adsorption column comprising a particulate matrix, applying a milk source to the matrix, and eluting the lactoferrin from the matrix with an elution buffer comprising about 0.3 to about 2.0 M sodium chloride. Any mammalian milk source may be used in the present processes, although the milk source may be a bovine milk source. The milk source may comprise whole milk, reduced fat milk, skim milk, whey, casein, or mixtures thereof.

The target protein may be lactoferrin, though other milk proteins, such as lactoperoxidases or lactalbumins, also may be isolated. The process may comprise the steps of establishing an expanded bed adsorption column comprising a particulate matrix, applying a milk source to the matrix, and eluting the lactoferrin from the matrix with about 0.3 to about 2.0M sodium chloride. The lactoferrin may be eluted with about 0.5 to about 1.0 M sodium chloride. The lactoferrin may be eluted with about 0.7 to about 0.9 M sodium chloride.

The expanded bed adsorption column can be any known in the art, such as those described in U.S. Pat. Nos. 7,812,138, 6,620,326, and 6,977,046, the disclosures of which are hereby incorporated by reference herein. A milk source may be applied to the column in an expanded mode, and the elution may be performed in either expanded or packed mode. The elution may be performed in an expanded mode. For example, the expansion ratio in the expanded mode may be about 1 to about 3, or about 1.3 to about 1.7. EBA technology is further described in international published application nos. WO 92/00799, WO 02/18237, WO 97/17132, which are hereby incorporated by reference in their entireties.

The isoelectric point of lactoferrin is approximately 8.9. Prior EBA methods of isolating lactoferrin use 200 mM sodium hydroxide as an elution buffer. Thus, the pH of the system rises to over 12, and the structure and bioactivity of lactoferrin may be comprised, by irreversible structural changes. It has now been discovered that a sodium chloride solution can be used as an elution buffer in the isolation of lactoferrin from the EBA matrix. The sodium chloride may have a concentration of about 0.3 M to about 2.0 M. The lactoferrin elution buffer may have a sodium chloride concentration of about 0.3 M to about 1.5 M, or about 0.5 m to about 1.0 M.

Lactoferrin for use in the composition of the present disclosure may be isolated through the use of radial chromatography or charged membranes, as would be familiar to the skilled artisan.

The lactoferrin that is used may be any lactoferrin isolated from whole milk and/or having a low somatic cell count, wherein “low somatic cell count” refers to a somatic cell count less than 200,000 cells/mL. By way of example, suitable lactoferrin is available from Tatua Co-operative Dairy Co. Ltd., in Morrinsville, New Zealand, from FrieslandCampina Domo in Amersfoort, Netherlands or from Fonterra Co-Operative Group Limited in Auckland, New Zealand.

Surprisingly, lactoferrin included herein maintains certain bactericidal activity even if exposed to a low pH (i.e., below about 7, and even as low as about 4.6 or lower) and/or high temperatures (i.e., above about 65° C., and as high as about 120° C.), conditions which would be expected to destroy or severely limit the stability or activity of human lactoferrin. These low pH and/or high temperature conditions can be expected during certain processing regimen for nutritional compositions of the types described herein, such as pasteurization. Therefore, even after processing regimens, lactoferrin has bactericidal activity against undesirable bacterial pathogens found in the human gut. The nutritional composition may comprise lactoferrin in an amount from about 25 mg/100 mL to about 150 mg/100 mL. Lactoferrin may be present in an amount from about 60 mg/100 mL to about 120 mg/100 mL. Lactoferrin may be present in an amount from about 85 mg/100 mL to about 110 mg/100 mL.

The nutritional composition(s) of the present disclosure may comprise choline. Choline is a nutrient that is essential for normal function of cells. It is a precursor for membrane phospholipids, and it accelerates the synthesis and release of acetylcholine, a neurotransmitter involved in memory storage. Moreover, though not wishing to be bound by this or any other theory, it is believed that dietary choline and docosahexaenoic acid (DHA) act synergistically to promote the biosynthesis of phosphatidylcholine and thus help promote synaptogenesis in human subjects. Additionally, choline and DHA may exhibit the synergistic effect of promoting dendritic spine formation, which is important in the maintenance of established synaptic connections. The nutritional composition(s) of the present disclosure may include about 40 mg choline per serving to about 100 mg per 8 oz. serving.

The nutritional composition may comprise a source of iron. The source of iron may be ferric pyrophosphate, ferric orthophosphate, ferrous fumarate or a mixture thereof and the source of iron may be encapsulated.

One or more vitamins and/or minerals may also be added in to the nutritional composition in amounts sufficient to supply the daily nutritional requirements of a subject. It is to be understood by one of ordinary skill in the art that vitamin and mineral requirements will vary, for example, based on the age of the subject. For instance, an infant may have different vitamin and mineral requirements than a child between the ages of one and thirteen years. Thus, the aspects are not intended to limit the nutritional composition to a particular age group but, rather, to provide a range of acceptable vitamin and mineral components.

The composition may optionally include, but is not limited to, one or more of the following vitamins or derivations thereof: vitamin B1 (thiamin, thiamin pyrophosphate, TPP, thiamin triphosphate, TTP, thiamin hydrochloride, thiamin mononitrate), vitamin B2 (riboflavin, flavin mononucleotide, FMN, flavin adenine dinucleotide, FAD, lactoflavin, ovoflavin), vitamin B3 (niacin, nicotinic acid, nicotinamide, niacinamide, nicotinamide adenine dinucleotide, NAD, nicotinic acid mononucleotide, NicMN, pyridine-3-carboxylic acid), vitamin B3-precursor tryptophan, vitamin B6 (pyridoxine, pyridoxal, pyridoxamine, pyridoxine hydrochloride), pantothenic acid (pantothenate, panthenol), folate (folic acid, folacin, pteroylglutamic acid), vitamin B12 (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D3, 1,25,-dihydroxyvitamin D), vitamin E (α-tocopherol, α-tocopherol acetate, α-tocopherol succinate, α-tocopherol nicotinate, α-tocopherol), vitamin K (vitamin K1, phylloquinone, naphthoquinone, vitamin K2, menaquinone-7, vitamin K3, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

The composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to the nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

The nutritional composition may contain between about 10 and about 50% of the maximum dietary recommendation for any given country, or between about 10 and about 50% of the average dietary recommendation for a group of countries, per serving of vitamins A, C, and E, zinc, iron, iodine, selenium, and choline. The nutritional composition may supply about 10-30% of the maximum dietary recommendation for any given country, or about 10-30% of the average dietary recommendation for a group of countries, per serving of B-vitamins. The levels of vitamin D, calcium, magnesium, phosphorus, and potassium in the nutritional product may correspond with the average levels found in milk. Other nutrients in the nutritional composition may be present at about 20% of the maximum dietary recommendation for any given country, or about 20% of the average dietary recommendation for a group of countries, per serving.

The nutritional composition of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg or soy), alpha lactalbumin and/or mono- and di-glycerides, and mixtures thereof. Other emulsifiers are readily apparent to the skilled artisan and selection of suitable emulsifier(s) will depend, in part, upon the formulation and final product

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, calcium disodium EDTA, and mixtures thereof.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof.

The nutritional compositions of the disclosure may provide minimal, partial or total nutritional support. The compositions may be nutritional supplements or meal replacements. The compositions may, but need not, be nutritionally complete. The nutritional composition of the disclosure may be nutritionally complete and contains suitable types and amounts of lipid, carbohydrate, protein, vitamins and minerals. The amount of lipid or fat typically can vary from about 2 to about 7 g/100 kcal. The amount of protein typically can vary from about 1 to about 5 g/100 kcal. The amount of carbohydrate typically can vary from about 8 to about 14 g/100 kcal.

The nutritional composition of the present disclosure may be a growing-up milk. Growing-up milks are fortified milk-based beverages intended for children over 1 year of age (typically from 1-6 years of age). They are not medical foods and are not intended as a meal replacement or a supplement to address a particular nutritional deficiency. Instead, growing-up milks are designed with the intent to serve as a complement to a diverse diet to provide additional insurance that a child achieves continual, daily intake of all essential vitamins and minerals, macronutrients plus additional functional dietary components, such as non-essential nutrients that have purported health-promoting properties.

The exact composition of an infant formula or a growing-up milk or other nutritional composition according to the present disclosure can vary from market-to-market, depending on local regulations and dietary intake information of the population of interest. Nutritional compositions according to the disclosure may consist of a milk protein source, such as whole or skim milk, plus added sugar and sweeteners to achieve desired sensory properties, and added vitamins and minerals. The fat composition is typically derived from the milk raw materials. Total protein can be targeted to match that of human milk, cow milk or a lower value. Total carbohydrate is usually targeted to provide as little added sugar, such as sucrose or fructose, as possible to achieve an acceptable taste. Typically, Vitamin A, calcium and Vitamin D are added at levels to match the nutrient contribution of regional cow milk. Otherwise, vitamins and minerals may be added at levels that provide approximately 20% of the dietary reference intake (DRI) or 20% of the Daily Value (DV) per serving. Moreover, nutrient values can vary between markets depending on the identified nutritional needs of the intended population, raw material contributions and regional regulations.

The pediatric subject may be a child or an infant. For example, the subject may an infant ranging in age from 0 to 3 months, about 0 to 6 months, 0 to 12 months, 3 to 6 months, or 6 to 12 months. The subject may alternatively be a child ranging in age from 1 to 13 years, 1 to 6 years or 1 to 3 years. The composition may be administered to the pediatric subject prenatally, during infancy, and during childhood.

### EXPERIMENTAL

### Experiment 1

Experiment 1 illustrates the effects of diets supplemented with GOS, PDS, and SAL had on the structure and function of gut microbiota.

Adult male C57bl/6 strains of Mus musculus (Charles Rivers Laboratories, Wilmington, Mass.) between 6-8 weeks of age were used in the study. After being received from the vendor, mice were placed on one of three experimental diets. Animals were given ad libitum access to water, and maintained on the experimental diets for 3 weeks. Experimental diets were: (1) Control diet (AIN-93G mouse chow); (2) AIN-93G supplemented with galactooligosaccharides (GOS 21.2 g/kg)+polydextrose (PDX 6.6 g/kg)+sialyllactose (SAL 2 g/kg); (3) AIN-93G supplemented with SAL.

Colon contents were removed via direct excision for metabolomic analysis, and the colon tissue was briefly washed in a PBS bath so as to not disturb the mucous layer. DNA was extracted from the medial section of the colon (^{˜}10 mg) using a Qiagen DNA Mini Kit, following manufacturer's instructions with slight modifications. Briefly, tissues were incubated for 45 min at 37° C. in lysozyme buffer (20 mg/ml lysozyme, 20 mM TrisHCL, 2 mM EDTA, 1.2% Triton-x, pH 8.0), then bead-beat for 150 sec with 0.7 mm zirconia beads. Samples were incubated at 56° C. for 2 hr with Buffer ATL and Proteinase K, then incubated at 56° C. for 30 min and 95° C. for 10 min upon addition of Buffer AL. Following this step, the Qiagen DNA Mini Kit isolation protocol was followed beginning with the ethanol step. DNA was quantified with the Qubit 2.0 Fluorometer (Life Technologies, Carlsbad, Calif.) using the dsDNA Broad Range Assay Kit. Samples were standardized to at least 5 ng/μl before being sent to the Molecular and Cellular Imaging Center (MCIC) in Wooster, Ohio for library preparation. The V4-V5 hypervariable region of the 16s rRNA gene was targeted in this study. To amplify and sequence the V4-V5 region, we used primers that contain a heterogeneity spacer in line with the targeted sequence. Four sets of spacers of different lengths were used to compensate for the low nucleotide diversity of the amplicons; since accurate base-calling on Illumina platforms and generation of high-quality data requires sequence diversity at each nucleotide position before the clustering occurs. For the targeted region, we used well-known universal primers that were modified to include degenerate bases of maximal inclusiveness (Sci Reports 25).

The amplicon libraries were sequenced at the MCIC using the MiSeq sequencing platform (Illumina) at a final concentration of 15.4 pM. A genomic library of well-known diversity previously sequenced in the lab was combined with the pool of amplicon libraries for the sequencing run (expected at 20%). The run was clustered to a density of 1131 k/mm² and the libraries were sequenced using 300PE MiSeq sequencing kit with the standard Illumina sequencing primers. Image analysis, base calling and data quality assessment were performed on the MiSeq Instrument.

Metabolomics: Frozen samples were shipped to Metabolon (Durham, N.C.) for processing using the automated MicroLab STAR system (Hamilton Company). Recovery standards were added prior to extraction process. Proteins were precipitated with methanol under vigorous shaking for 2 min followed by centrifugation. The resulting extract was dividing into five fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. Organic solvent was removed with a TurboVap (Zymark) and extracts were stored overnight under nitrogen prior to analysis. In addition to the recovery standards and internal standards, quality control samples, including a pool of human plasma characterized by Metabolon to have known compounds, a pool of aliquots from every sample in this study, pure water, and solvent extract were used. Instrument variability was determined by calculating the median relative standard deviation for the recovery and internal standards. Process variability was determined by calculating the median relative standard deviation for all endogenous metabolites present in 100% of the pooled aliquots from every sample. Experimental samples were randomized across the platform run with QC samples spaced between every third sample injection.

Raw data was extracted, peak-identified and QC processed using Metabolon's hardware and software. Compounds were identified by comparison to a library of over 3,300 purified standards to recurrent unknown entities. Biochemical identifications were based on retention index within a narrow RI window of the proposed identification, accurate mass match to the library (+/−10 ppm), and the MS/MS forward and reverse scores between the experimental data and authentic standards. Peaks were quantified using area-under-the-curve. Values were normalized to account for variability across run days, and the intensity values were rescaled to set the median equal to 1.

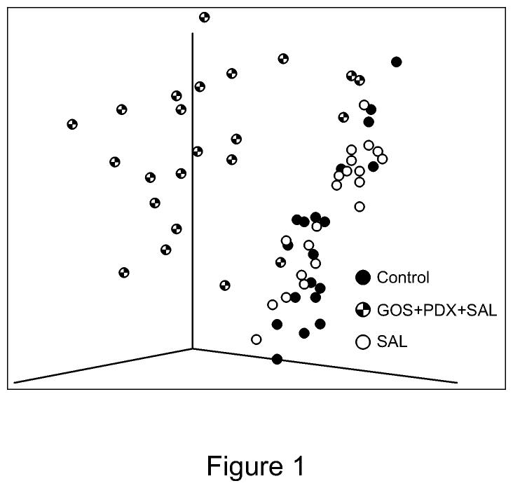

As shown in FIG. 1, the microbiome of mice receiving diets of GOS+PDX+SAL clusters separately versus Control-fed mice or SAL-fed mice. This indicates that the combination of GOS+PDX+SAL uniquely impacts the microbiome profile after 3 weeks of feeding. This effect was not achieved in the group fed only SAL.

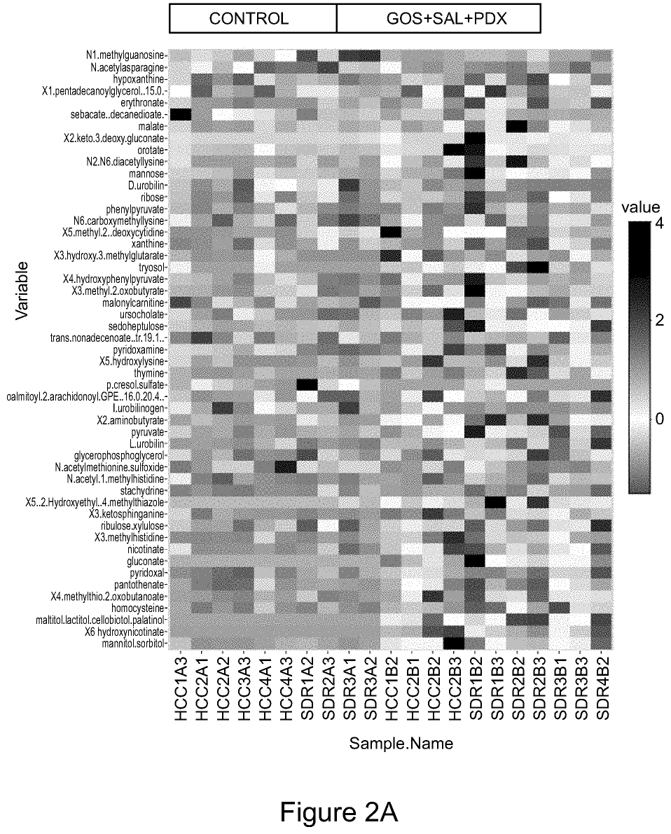

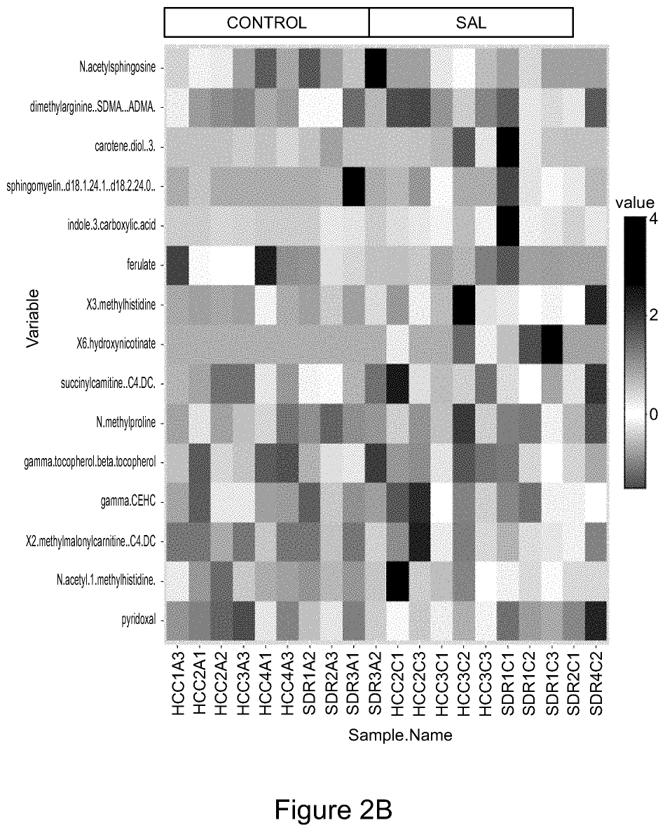

Furthermore, as shown in FIGS. 2A and 2B, the heat maps of fecal metabolites were different in the mice receiving diets of GOS+PDX+SAL, compared to the Control group or mice supplemented with SAL only. All selected metabolites were confirmed as significant by Random Forest and Boruta feature selection, when applied to all three groups simultaneously. The novel combination of GOS+PDX+SAL significantly changed the concentration of a higher number of metabolites compared to the SAL-only treatment. Further, the combination of GOS+PDX+SAL resulted in increased metabolite levels of polyunsaturated fatty acids (such as eicosapentaenoate and docosapentaenoate) and endocannabinoids (such as oleoyl ethanolamide, palmitoyl ethanolamide, and stearoyl ethanolamide), compared to the Control group. Polyunsaturated fatty acids and endocannabinoids have previously been shown to have anti-inflammatory abilities. The data produced in Experiment 1 therefore indicates increased bioactivity and a distinctive mechanism of action of the GOS+PDX+SAL mix.

### EXAMPLES

Examples are provided to illustrate some aspects of the nutritional composition of the present disclosure but should not be interpreted as any limitation thereon. Other aspects within the scope of the claims herein will be apparent to one skilled in the art from the consideration of the specification or practice of the nutritional composition or methods disclosed herein. It is intended that the specification, together with the example, be considered to be exemplary only, with the scope and spirit of the disclosure being indicated by the claims which follow the example.

### Formulation Example 1

| | per 100 kcal | |
|---|---|---|
| Nutrient/Lipid | Minimum | Maximum |
| Protein (g) | 1 | 7 |
| Fat (g) | 1 | 10 |
| Carbohydrates (g) | 5 | 25 |
| DHA (mg) | 5 | 100 |
| GOS (g) | 0.1 | 1.0 |
| PDX (g) | 0.1 | 0.5 |
| LGG (CFU) | 1 × 10⁴ | 1.5 × 10¹⁰ |
| Milk oligosaccharides | 0.005 | 1 |
| (e.g. sialyllactose) (g) | | |
| MFGM | 0.15 | 1.5 |
| Vitamin A (IU) | 134 | 921 |
| Vitamin D (IU) | 22 | 126 |
| Vitamin E (IU) | 0.8 | 5.4 |
| Vitamin K (mcg) | 2.9 | 18 |
| Thiamin (mcg) | 63 | 328 |
| Riboflavin (mcg) | 68 | 420 |
| Vitamin B6 (mcg) | 52 | 397 |
| Vitamin B12 (mcg) | 0.2 | 0.9 |
| Niacin (mcg) | 690 | 5881 |
| Folic acid (mcg) | 8 | 66 |
| Panthothenic acid (mcg) | 232 | 1211 |
| Biotin (mcg) | 1.4 | 5.5 |
| Vitamin C (mg) | 4.9 | 24 |
| | per 100 kcal | |
| Nutrient/Lipid | Minimum | Minimum |
| Choline (mg) | 4.9 | 43 |
| Calcium (mg) | 68 | 297 |
| Phosphorus (mg) | 54 | 210 |
| Magnesium (mg) | 4.9 | 34 |
| Sodium (mg) | 24 | 88 |
| Potassium (mg) | 82 | 346 |
| Chloride (mg) | 53 | 237 |
| Iodine (mcg) | 8.9 | 79 |
| Iron (mg) | 0.7 | 2.8 |
| Zinc (mg) | 0.7 | 2.4 |
| Manganese (mcg) | 7.2 | 41 |
| Copper (mcg) | 16 | 331 |

### Formulation Example 2

| | Present (mg/ml) | | | |
|---|---|---|---|---|
| Nutrient/Lipid | I | II | III | IV |
| Human milk oligosaccharide or a | ✓ | ✓ | 0.5-10 | 0.5-10 |
| precursor thereof | | | | |
| Milk fat globule membrane (MFGM) | ✓ | X | ✓ | X |
| supplied by an enriched milk | | | | |
| product formed from any milk source | | | | |
| Milk fat globule membrane (MFGM) supplied | X | ✓ | X | ✓ |
| by an enriched milk product formed from | | | | |
| a bovine milk source | | | | |
| Galacto-oligosaccharide (GOS) and/or | ✓ | ✓ | ✓ | ✓ |
| polydextrose (PDX) | | | | |

Key: √ = present; 0.5-10 = present at specified amount (mg/ml); X = not present

### Formulation Example 3

| | Present (mg/ml) | | | |
|---|---|---|---|---|
| Nutrient/Lipid | V | VI | VII | VIII |
| Human milk oligosaccharide or a precursor | ✓ | ✓ | 0.5-10 | 0.5-10 |
| thereof comprising at least one or more of | | | | |
| 2′-fucosyllactose, 3′-fucosyllactose, | | | | |
| 3′sialyllactose, 6'sialyllactose, lacto-N-biose, | | | | |
| lacto-N-neotetraose, lacto-N-tetraose, or | | | | |
| combinations thereof | | | | |
| Milk fat globule membrane (MFGM) | ✓ | X | ✓ | X |
| supplied by an enriched milk product formed | | | | |
| from any milk source | | | | |
| Milk fat globule membrane (MFGM) supplied | X | ✓ | X | ✓ |
| by an enriched milk product formed | | | | |
| from a bovine milk source | | | | |
| Galacto-oligosaccharide (GOS) | ✓ | ✓ | ✓ | ✓ |
| and/or polydextrose (PDX) | | | | |

Key: ✓ = present; 0.5-10 = present at specified amount (mg/ml); X = not present

### Formulation Example 4

| | Present (mg/ml) | | | |
|---|---|---|---|---|
| Nutrient/Lipid | IX | X | XI | XII |
| Human milk oligosaccharide or a | ✓ | ✓ | X | X |
| precursor thereof | | | | |
| Human milk oligosaccharide or a | X | X | ✓ | ✓ |
| precursor thereof | | | | |
| comprising at least one or more of | | | | |
| 2′-fucosyllactose, 3′-fucosyllactose, | | | | |
| 3′sialyllactose, 6′sialyllactose, lacto-N- | | | | |
| biose, lacto-N-neotetraose, lacto- | | | | |
| N-tetraose, or combinations thereof | | | | |
| Milk fat globule membrane (MFGM) | 1.5-7.5 | X | 1.5-7.5 | X |
| supplied by an enriched milk | | | | |
| product formed from any milk source | | | | |
| Milk fat globule membrane (MFGM) | X | 1.5-7.5 | X | 1.5-7.5 |
| supplied by an | | | | |
| enriched milk product formed from a | | | | |
| bovine milk source | | | | |
| Galacto-oligosaccharide (GOS) and/or | ✓ | ✓ | ✓ | ✓ |
| polydextrose (PDX) | | | | |

Key: ✓ = present; 1.5-7.5 = present at specified amount (mg/ml); X = not present

### Formulation Example 5

| | Present (mg/ml) | | | |
|---|---|---|---|---|
| Nutrient/Lipid | XIII | XIV | XV | XVI |
| Human milk oligosaccharide or a | 0.5-10 | 0.5-10 | X | X |
| precursor thereof | | | | |
| Human milk oligosaccharide or a | X | X | 0.5-10 | 0.5-10 |
| precursor thereof | | | | |
| comprising at least one or more of | | | | |
| 2′-fucosyllactose, 3′- | | | | |
| fucosyllactose, 3′sialyllactose, | | | | |
| 6′sialyllactose, lacto-N-biose, | | | | |
| lacto-N-neotetraose, | | | | |
| lacto-N-tetraose, | | | | |
| or combinations thereof | | | | |
| Milk fat globule membrane | 1.5-7.5 | X | 1.5-7.5 | X |
| (MFGM) supplied by an | | | | |
| enriched milk product formed | | | | |
| from any milk source | | | | |
| Milk fat globule membrane | X | 1.5-7.5 | X | 1.5-7.5 |
| (MFGM) supplied by an | | | | |
| enriched milk product formed | | | | |
| from a bovine milk source | | | | |
| Galacto-oligosaccharide | ✓ | ✓ | ✓ | ✓ |
| (GOS) and/or polydextrose (PDX) | | | | |

Key: ✓ = present; 0.5-10/1.5-7.5 = present at specified amount (mg/ml); X = not present

1-46. (canceled)

47. A nutritional composition comprising:
(i) at least one human milk oligosaccharide or a precursor thereof; and
(ii) galacto-oligosaccharide (GOS) and/or polydextrose (PDX),
wherein the nutritional composition is adapted for administration to an infant delivered by C-section.

48. The composition of claim 47, wherein the at least one human milk oligosaccharide comprises one or more of 2′-fucosyllactose, 3′-fucosyllactose, 3′sialyllactose, 6′sialyllactose, lacto-N-biose, lacto-N-neotetraose, lacto-N-tetraose, or combinations thereof.

49. The composition of claim 48, wherein the at least one human milk oligosaccharide comprises 3′sialyllactose, 6′sialyllactose, or a combination thereof.

50. The composition of claim 47, wherein the at least one human milk oligosaccharide is present at a concentration ranging from about 0.5 mg/ml to about 10 mg/ml of the nutritional composition.

51. The composition of claim 47, wherein the GOS and/or PDX is present in an amount of about 1 mg/ml to about 6 mg/ml.

52. The composition of claim 47, further comprising milk fat globule membrane (MFGM).

53. The composition of claim 52, wherein the MFGM is present in an amount of about 1.5 mg/ml to about 7.5 mg/ml of the nutritional composition.

54. The composition of claim 52, wherein the MFGM is supplied by an enriched milk product formed from a bovine milk source.

55. The composition of claim 47, wherein the GOS and/or PDX is present in an amount of about 1 mg/ml to about 6 mg/ml.

56. The composition of claim 47, wherein the nutritional composition is a synthetic nutritional composition.

57. The composition of claim 47, wherein the at least one human milk oligosaccharide comprises one or more of 2′-fucosyllactose, 3′-fucosyllactose, 3′sialyllactose, 6′sialyllactose, lacto-N-biose, lacto-N-neotetraose, lacto-N-tetraose, or combinations thereof, wherein the human milk oligosaccharide is present at a concentration ranging from about 0.5 mg/ml to about 10 mg/ml of the nutritional composition, and
wherein the GOS and PDX is present in an amount of about 1 mg/ml to about 6 mg/ml.

58. The composition of claim 47, wherein the at least one human milk oligosaccharide comprises 3′sialyllactose, 6′sialyllactose, or a combination thereof, wherein the human milk oligosaccharide is present at a concentration ranging from about 0.5 mg/ml to about 10 mg/ml of the nutritional composition, and
wherein the GOS and PDX is present in an amount of about 1 mg/ml to about 6 mg/ml.

59. The composition of claim 47, wherein the at least one human milk oligosaccharide comprises one or more of 2′-fucosyllactose, 3′-fucosyllactose, 3′sialyllactose, 6′sialyllactose, lacto-N-biose, lacto-N-neotetraose, lacto-N-tetraose, or combinations thereof, wherein the human milk oligosaccharide is present at a concentration ranging from about 0.5 mg/ml to about 10 mg/ml of the nutritional composition,
wherein the GOS and PDX is present in an amount of about 1 mg/ml to about 6 mg/ml, and
wherein the nutritional composition further comprises milk fat globule membrane (MFGM).

60. The composition of claim 47, wherein the at least one human milk oligosaccharide comprises 3′sialyllactose, 6′sialyllactose, or a combination thereof, wherein the human milk oligosaccharide is present at a concentration ranging from about 0.5 mg/ml to about 10 mg/ml of the nutritional composition,
wherein the GOS and PDX is present in an amount of about 1 mg/ml to about 6 mg/ml, and
wherein the nutritional composition further comprises milk fat globule membrane (MFGM).

61. The composition of claim 47, wherein the at least one human milk oligosaccharide comprises one or more of 2′-fucosyllactose, 3′-fucosyllactose, 3′sialyllactose, 6′sialyllactose, lacto-N-biose, lacto-N-neotetraose, lacto-N-tetraose, or combinations thereof, wherein the human milk oligosaccharide is present at a concentration ranging from about 0.5 mg/ml to about 10 mg/ml of the nutritional composition,
wherein the GOS and PDX is present in an amount of about 1 mg/ml to about 6 mg/ml, and
wherein the nutritional composition further comprises milk fat globule membrane (MFGM) in an amount of about 1.5 mg/ml to about 7.5 mg/ml of the nutritional composition.

62. The composition of claim 47, wherein the at least one human milk oligosaccharide comprises one or more of 2′-fucosyllactose, 3′-fucosyllactose, 3′sialyllactose, 6′sialyllactose, lacto-N-biose, lacto-N-neotetraose, lacto-N-tetraose, or combinations thereof, wherein the human milk oligosaccharide is present at a concentration ranging from about 0.5 mg/ml to about 10 mg/ml of the nutritional composition,
wherein the GOS and PDX is present in an amount of about 1 mg/ml to about 6 mg/ml, and
wherein the nutritional composition further comprises milk fat globule membrane (MFGM) supplied by an enriched milk product formed from a bovine milk source.

63. The composition of claim 47, wherein the at least one human milk oligosaccharide comprises one or more of 2′-fucosyllactose, 3′-fucosyllactose, 3′sialyllactose, 6′sialyllactose, lacto-N-biose, lacto-N-neotetraose, lacto-N-tetraose, or combinations thereof, wherein the human milk oligosaccharide is present at a concentration ranging from about 0.5 mg/ml to about 10 mg/ml of the nutritional composition,
wherein the GOS and PDX is present in an amount of about 1 mg/ml to about 6 mg/ml,
wherein the nutritional composition further comprises milk fat globule membrane (MFGM) present in an amount of about 1.5 mg/ml to about 7.5 mg/ml of the nutritional composition, and
wherein the MFGM is supplied by an enriched milk product formed from a bovine milk source.

64. The composition of claim 47, wherein the at least one human milk oligosaccharide comprises 3′sialyllactose, 6′sialyllactose, or a combination thereof, wherein the human milk oligosaccharide is present at a concentration ranging from about 0.5 mg/ml to about 10 mg/ml of the nutritional composition,
wherein the GOS and PDX is present in an amount of about 1 mg/ml to about 6 mg/ml, and
wherein the nutritional composition further comprises milk fat globule membrane (MFGM) present in an amount of about 1.5 mg/ml to about 7.5 mg/ml of the nutritional composition.

65. The composition of claim 47, wherein the at least one human milk oligosaccharide comprises 3′sialyllactose, 6′sialyllactose, or a combination thereof, wherein the human milk oligosaccharide is present at a concentration ranging from about 0.5 mg/ml to about 10 mg/ml of the nutritional composition,
wherein the GOS and PDX is present in an amount of about 1 mg/ml to about 6 mg/ml, and
wherein the nutritional composition further comprises milk fat globule membrane (MFGM) supplied by an enriched milk product formed from a bovine milk source.

66. The composition of claim 47, wherein the at least one human milk oligosaccharide comprises 3′sialyllactose, 6′sialyllactose, or a combination thereof, wherein the human milk oligosaccharide is present at a concentration ranging from about 0.5 mg/ml to about 10 mg/ml of the nutritional composition,
wherein the GOS and PDX is present in an amount of about 1 mg/ml to about 6 mg/ml,
wherein the nutritional composition further comprises milk fat globule membrane (MFGM) present in an amount of about 1.5 mg/ml to about 7.5 mg/ml of the nutritional composition, and
wherein the MFGM is supplied by an enriched milk product formed from a bovine milk source.
